(19) United States
(12) Patent Application Publication
(10) Pub. No.: US 2010/0184979 A1
(43) Pub. Date: Jul. 22, 2010

(54) NEW PROCESS FOR PREPARING 2-(3--PHENYL)-2-METHYLPROPRIONIC ACID

(75) Inventors: Reda G. HANNA, Allentown, PA (US); Elizabeth SECORD, Langhorne, PA (US); Bao-Guo HUANG, Bridgewater, NJ (US); Matthew R. POWERS, Easton, PA (US)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/639,507

(22) Filed: Dec. 16, 2009

Related U.S. Application Data
(63) Continuation of application No. PCT/US2008/067963, filed on Jun. 24, 2008.
(60) Provisional application No. 60/947,256, filed on Jun. 29, 2007.

(51) Int. Cl. C07D 239/47

(52) U.S. Cl. 544/317

(57) ABSTRACT

This invention is directed to a process for preparing 2-(3-{6-[2-(2,4-dichloro-phenyl)-ethylamino]-2-methoxy-pyrimidin-4-yl}-phenyl)-2-methyl-propionic acid.

### FIELD OF THE INVENTION

This invention is directed to a process for preparing 2-(3-{6-[2-(2,4-dichlorophenyl)-ethylamino]-2-methoxypyrimidin-4-yl}-phenyl)-2-methylpropionic acid.

### BACKGROUND OF THE INVENTION

Patent application WO 2006044732 (hereinafter the '732 application), hereby incorporated by reference, discloses pyrimidines of Formula (I),

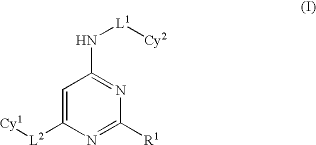

wherein R¹, L¹, L², Cy¹ and Cy² are as defined therein, their preparation, pharmaceutical compositions containing these compounds, and their pharmaceutical use in the treatment of disease states capable of being modulated by the inhibition of the prostaglandin D2 receptor, such as asthma [Arimura A, Yasui K, Kishino J, Asanuma F, Hasegawa H, Kakudo S, Ohtani M, Arita H, Prevention of allergic inflammation by a novel prostaglandin receptor antagonist, S-5751, J Pharmacol Exp Ther. 298(2), 411-9, 2001], allergic rhinitis [Jones, T. R., Savoie, C., Robichaud, A., Sturino, C., Scheigetz, J., Lachance, N., Roy, B., Boyd, M., Abraham, W., Studies with a DP receptor antagonist in sheep and guinea pig models of allergic rhinitis, Am. J. Resp. Crit. Care Med. 167, A218, 2003; and Arimura A, Yasui K, Kishino J, Asanuma F, Hasegawa H, Kakudo S, Ohtani M, Arita H, Prevention of allergic inflammation by a novel prostaglandin receptor antagonist, S-5751. J. Pharmacol. Exp. Ther. 298(2), 411-9, 2001], and allergic conjunctivitis and allergic dermatitis [Arimura A, Yasui K, Kishino J, Asanuma F, Hasegawa H, Kakudo S, Ohtani M, Arita H, Prevention of allergic inflammation by a novel prostaglandin receptor antagonist, S-5751. J. Pharmacol. Exp. Ther. 298(2), 411-9, 2001; and Torisu K, Kobayashi K, Iwahashi M, Nakai Y, Onoda T, Nagase T, Sugimoto I, Okada Y, Matsumoto R, Nanbu F, Ohuchida S, Nakai H, Toda M, Discovery of a new class of potent, selective, and orally active prostaglandin D₂ receptor antagonists, Bioorg. & Med. Chem. 12, 5361-5378, 2004].

The '732 application discloses a process for preparing pyrimidines of Formula (I) wherein L² is a bond, by reacting a corresponding compound of Formula (V), wherein X² is a halogen, preferably chlorine, or a triflate group, with a boronic acid of Formula (VI), or a boronic acid pinacol ester of formula (XVII) in the presence of cesium carbonate and a complex metal catalyst such as tetrakis(triphenylphosphine)palladium (0) or pddf

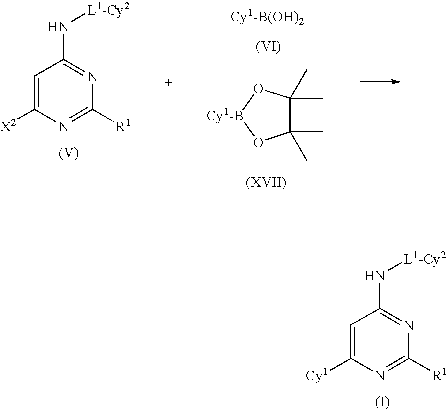

The '732 application further discloses that the compound of Formula (V) wherein X² is chlorine may be prepared by coupling a corresponding dichloropyrimidine of Formula (X) with a corresponding amine of Formula (IV), in the presence of a suitable base, such as sodium bicarbonate.

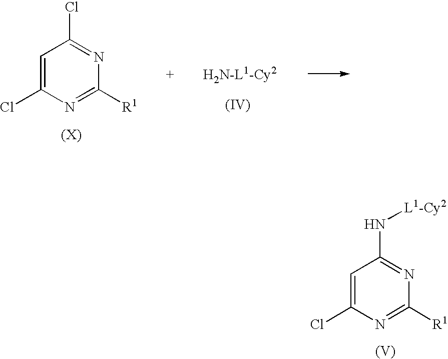

The '732 application more particularly discloses 2-(3-{6-[2-(2,4-dichlorophenyl)-ethylamino]-2-methoxypyrimidin-4-yl}-phenyl)-2-methylpropionic acid of formula (A),

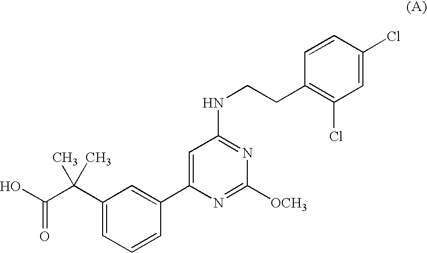

and its preparation as shown in Scheme I.

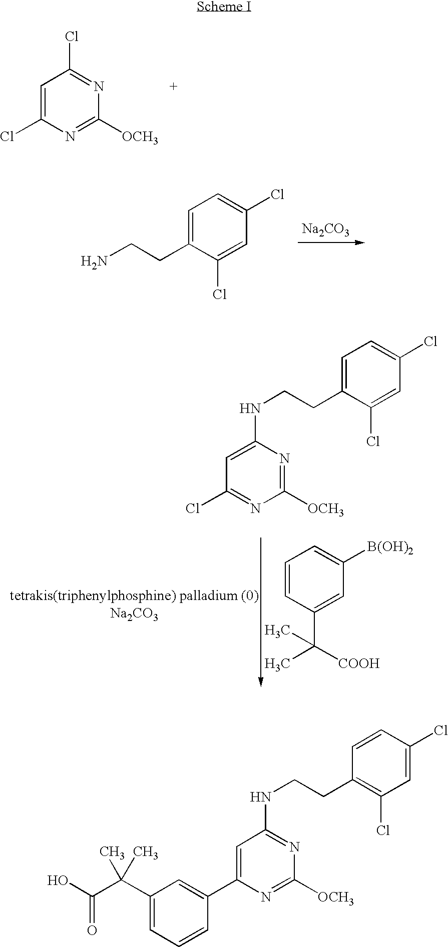

The '732 application also discloses an alternative process for preparing pyrimidines of Formula (I) by reacting a corresponding compound of Formula (III), wherein X¹ is a halogen, particularly chlorine, or a triflate group, with a corresponding amine of Formula (IV), in the a suitable base, such as sodium bicarbonate, in an inert solvent, such as 1-methyl-2-pyrrolidinone, and at a temperature at about 160° C.

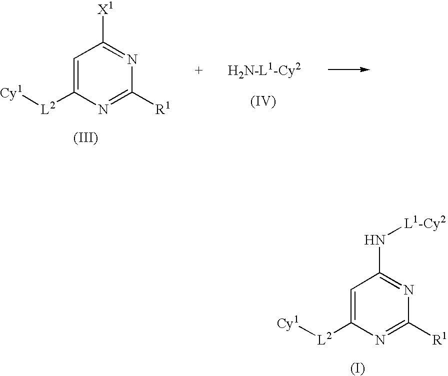

More particularly, Example 43(b) of the '732 application exemplifies the synthesis of [2-methoxy-6-(3-methoxy-phenyl)-pyrimidin-4-yl]-[2-(4-nitro-phenyl)-ethyl]-amine by such alternative process, as shown in Scheme II below:

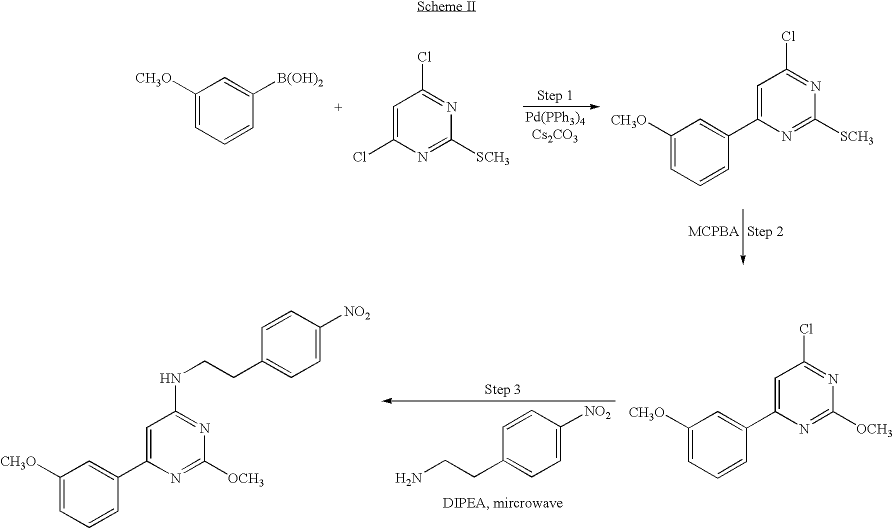

Patent application WO 2007047378 (hereinafter '378 application) also specifically discloses dihydrogen phosphate salt of 2-(3-{6-[2-(2,4-dichlorophenyl)-ethylamino]-2-methoxypyrimidin-4-yl}-phenyl)-2-methylpropionic acid, and its preparation. However, the dihydrogen salt prepared in THF according to the procedures described therein contains about 1400-1600 ppm residual THF, which exceeds the limit of International Conference on Harmonization (ICH) guideline of 700 ppm. Furthermore, recrystallization from acetone is not practical for large scale preparation due to the low solubility.

A process for the preparation of a pharmaceutical active ingredient used in a medicament on an industrial scale has to fulfill various requirements. The process and the obtained product have to be in line with the regulatory requirements and have to be reproducible and validated. In particular, regulatory authorities stipulate a precise degree of purity of the obtained drug substance and an acceptable level (e.g. single digit ppm) for heavy metals such as Pd or Ni. On the other hand, a process performed on an industrial scale for preparing a marketed product should of course be as simple, cost and labor effective as possible. If possible, it should thus avoid the use of expensive starting materials, physiologically unacceptable toxic materials, difficult technical operations, long reaction time, or multiple procedural steps.

2-(3-{6-[2-(2,4-Dichloro-phenyl)-ethylamino]-2-methoxy-pyrimidin-4-yl}-phenyl)-2-methyl-propionic acid prepared by following the procedures described in the '732 application requires chromatographic purification, which is not desirable. In addition, it is also not desirable to remove the residual Palladium catalyst in the last step of the process.

Furthermore, the procedures described in Example 43(b) of the '732 application utilize a high amount (5 mol %) of unstable catalyst, palladium tetrakis(triphenyl-phosphine). In addition, the intermediate and product formed therein also need to be purified by chromatography. Some steps in the process also require long reaction time or use of microwave equipment, which lead to a very low time-space yield and a technical difficulty for large scale operation.

Thus, there is a need for a simpler and improved process for manufacturing the compound of formula A. The present invention satisfies this need by providing such a process.

### SUMMARY OF THE INVENTION

The present invention is directed to a process for preparing 2-(3-{6-[2-(2,4-dichlorophenyl)-ethylamino]-2-methoxypyrimidin-4-yl}-phenyl)-2-methylpropionic acid of formula (A),

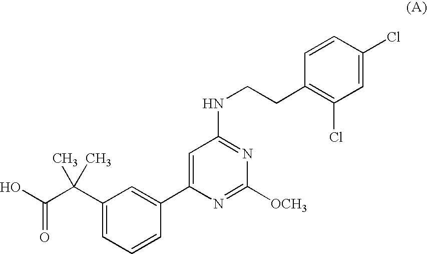

comprising:
(1) coupling 4,6-dichloro-2-methoxypyrimidine with 3-(1-carboxy-1-methyl-ethyl)-phenyl boronic acid in the presence of a suitable Palladium catalyst to provide 2-[3-(6-chloro-2-hydroxy-pyrimidin-4-yl)-phenyl]-2-methylpropionic acid,
(2) coupling 2-[3-(6-chloro-2-hydroxy-pyrimidin-4-yl)-phenyl]-2-methyl-propionic acid with 2,4-dichlorophenethylamine to provide 2-(3-{6-[2-(2,4-dichlorophenyl)-ethylamino]-2-methoxy-pyrimidin-4-yl}-phenyl)-2-methylpropionic acid.

Another aspect of the invention is the compound 2-[3-(6-chloro-2-hydroxypyrimidin-4-yl)-phenyl]-2-methylpropionic acid, or a salt thereof.

The present invention is more fully discussed with the aid of the following figures and detailed description below.

### BRIEF DESCRIPTION OF THE DRAWINGS

FIG. 1 is an HPLC spectrum of 2-(3-{6-[2-(2,4-dichloro-phenyl)-ethylamino]-2-methoxy-pyrimidin-4-yl}-phenyl)-2-methyl-propionic acid prepared by the process of the invention.

### DETAILED DESCRIPTION OF THE INVENTION

The present invention will be better appreciated by reference to the following detailed description.

### Definitions and Abbreviations

As used above, and throughout the description of the invention, the following abbreviations, unless otherwise indicated, shall be understood to have the following meanings.
DME 1,2-dimethoxyethane
g gram
h hour
HCl Hydrochloric acid
K₂CO₃ Potassium carbonate
Na₂CO₃ Sodium carbonate
Cs₂CO₃ Cesium carbonate
K₃PO₄ Potassium Phosphate
Na₃PO₄ Sodium Phosphate
NaOH Sodium hydroxide
KOH Potassium hydroxide
mg milligram
min minute
mL milliliter
n-BuOAc n-butyl acetate
pddf 1,1′-Bis(diphenylphosphino)ferrocene-palladium(II) dichloride dichloromethane complex
Pd(OAc)₂ Palladium (II) acetate
Pd(PPh₃)₄ tetrakis(triphenylphosphine)palladium (0)
PPh₃ triphenylphosphine
ppm parts per million
TMT trithiocyanuric acid
THF tetrahydrofuran
˜ approximately

As used above, and throughout the description of the invention, the following terms, unless otherwise indicated, shall be understood to have the following meanings

“Suitable Palladium catalyst” includes, for example, Pd, PdCl₂, Pd(OAc)₂, or pddf.
“Suitable base” includes inorganic base, for example, Na₂CO₃, K₂CO₃, Cs₂CO₃, K₃PO₄, KOH or NaOH, more particularly K₂CO₃, and organic base, for example, triethylamine or diisopropyl ethylamine.
“Suitable solvent system” include organic solvent, for example, alcohol, ethyl acetate, THF, DME, toluene, etc., or a mixture of water soluble organic solvent with water, for example, a mixture of DME and water, THF and water, or alcohol and water, more particularly a mixture of DME and water.

### PARTICULAR EMBODIMENTS OF THE INVENTION

In a particular embodiment of the invention, the first step coupling reaction is carried out in the presence of Pd, PdCl₂, Pd(PPh₃)₄, pddf, or Pd(OAc)₂; more particularly Pd(OAc)₂.

In another particular embodiment of the invention, the first step coupling reaction is carried out in the presence of Pd(OAc)₂ and PPh₃.

In another particular embodiment of the invention, the first step coupling reaction is carried out in the presence of about 0.01 mol % to about 0.5 mol % of Pd(OAc)₂ and about 0.02 mol % to about 1.0 mol % PPh₃, more particularly, about 0.1 mol % of Pd(OAc)₂ and about 0.2 mol % PPh₃.

In another particular embodiment of the invention, the first step coupling reaction is carried out by adding 3-(1-carboxy-1-methyl-ethyl)-phenyl boronic acid to 4,6-dichloro-2-methoxypyrimidine slowly over a period of time, preferably about 2-6 hrs, thus, significantly reducing the over reaction byproduct and thermal decomposition of boronic acid.

In another particular embodiment of the invention, the first step coupling reaction is carried out in the presence of a suitable base.

In another particular embodiment of the invention, the first step coupling reaction is carried out in the presence of a base, wherein the amount of the base is in the range of about 2-4 equivalents, more particularly, about 3.0 equivalents.

In another particular embodiment of the invention, the first step coupling reaction is carried out using about 1.0-2.0 equivalents of 4,6-dichloro-2-methoxypyrimidne, more particularly about 1.2 equivalents.

In another particular embodiment of the invention, the first step coupling reaction is carried out in the presence of a suitable solvent system.

In another particular embodiment of the invention, the first step coupling product, 2-[3-(6-chloro-2-hydroxy-pyrimidin-4-yl)-phenyl]-2-methylpropionic acid, is purified by:
removing excess 4,6-dichloro-2-methoxypyrimidne by a phase separation, adjusting pH of the phase containing 2-[3-(6-chloro-2-hydroxy-pyrimidin-4-yl)-phenyl]-2-methylpropionic acid to about 6.5 to about 7.5, more particularly about 7.2, and
extracting the phase containing 2-[3-(6-chloro-2-hydroxy-pyrimidin-4-yl)-phenyl]-2-methylpropionic acid with a water-immiscible organic solvent, for example, n-butyl acetate, ethyl acetate or toluene.

In another particular embodiment of the invention, wherein the Palladium catalyst is removed from 2-[3-(6-chloro-2-hydroxy-pyrimidin-4-yl)-phenyl]-2-methyl-propionic acid prior to the second step, for example, by treating 2-[3-(6-chloro-2-hydroxy-pyrimidin-4-yl)-phenyl]-2-methyl-propionic acid with TMT and charcoal, in an organic solvent, such as n-butyl acetate at about 50-70° C., particularly, at about 70° C.

In another particular embodiment of the invention, the first step product, 2-[3-(6-chloro-2-hydroxy-pyrimidin-4-yl)-phenyl]-2-methyl-propionic acid, is used directly in the second step as a solution.

In another particular embodiment of the invention, the second step is carried out in water.

In another particular embodiment of the invention, the second step product, 2-(3-{6-[2-(2,4-dichlorophenyl)-ethylamino]-2-methoxypyrimidin-4-yl}-phenyl)-2-methylpropionic acid, is purified by recrystallization from an organic solvent and water, for example, THF/water or DME/water, more particularly, DME/water.

In another particular embodiment of the invention, the compound of formula (A) is converted into dihydrogen phosphate salt thereof in methanol.

The present invention offers an improved process for preparing 2-(3-{6-[2-(2,4-dichloro-phenyl)-ethylamino]-2-methoxypyrimidin-4-yl}-phenyl)-2-methylpropionic acid in high yields and high purities yet requiring no expensive and time consuming column chromatographic purification as used in the '732 application. The present process uses a less amount of and more stable catalyst. A simpler phase separation is used to separate the step-1 product from excess reagents, and the product purification is achieved by a pH adjustment. The residual Pd removal is also reduced to the desired level (5 ppm) in step-1, thus avoiding handling the problem in the final stage since the final product is less soluble in common organic solvents, making it harder to operate. Furthermore, the step-1 product is concatenated into step-2 reaction without isolation. Overall, the present invention provides a much more efficient process for preparing the desired product in a high purity (>99% pure by HPLC) and high time-space yield. The salt formation carried out in methanol instead of THF as disclosed in the '378 application also eliminates the residual solvent issue.

### EXAMPLES

The present invention may be better understood by reference to the following non-limiting Examples, which are exemplary of the invention. They should in no way be construed, however, as limiting the broad scope of the invention.

### Example 1

### 2-(3-{6-[2-(2,4-Dichlorophenyl)-ethylamino]-2-methoxypyrimidin-4-yl}-phenyl)-2-methylpropionic acid

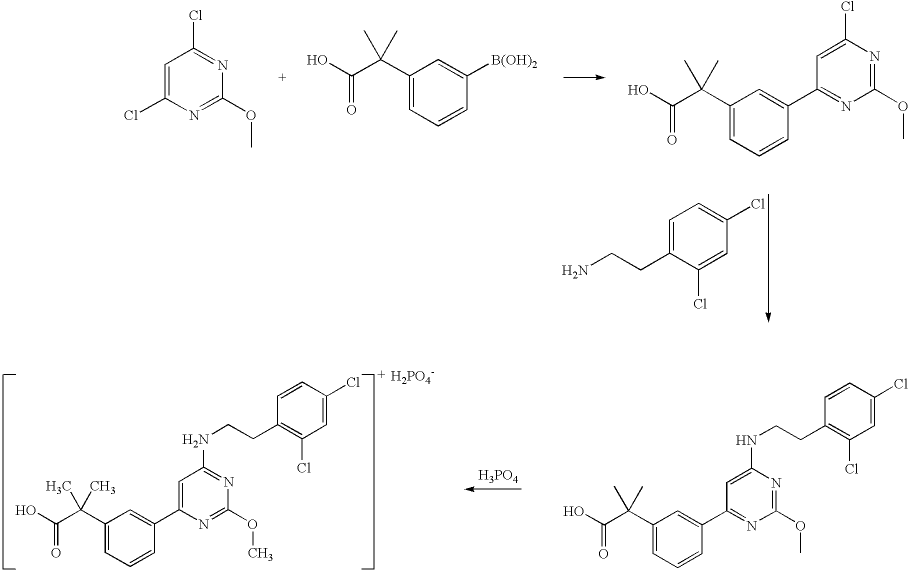

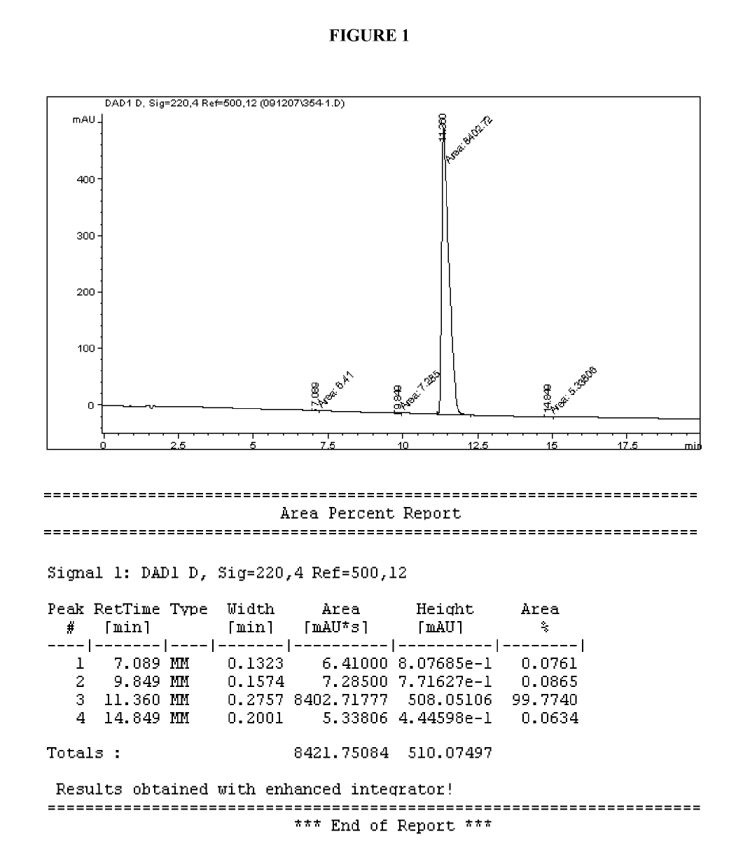

Step 1: To an 1-L reactor are added 4,6-dichloro-2-methoxy-pyrimidine (50.1 g), K₂CO₃ (96.4 g), Pd(OAc)₂ (48 mg, 0.1 mol %) and PPh₃ (120 mg, 0.2 mol %) in DME/water (100 mL/200 mL). The mixture is heated to 50° C., while 3-(1-carboxy-1-methyl-ethyl)-phenyl boronic acid (51.6 g, 94 wt % purity) in DME (50 mL)/water (100 mL) is added over 2 h via a metering pump. The reaction is held at 50° C. for an additional hour. The mixture is cooled to 25° C. and toluene (250 mL) is added. The mixture is stirred for 15 min and the layers are separated. To the aqueous layer is added n-BuOAc (300 mL) and pH is adjusted to 7.2 with 4 M aqueous HCl (˜190 mL). The aqueous layer is extracted with n-BuOAc (2×300 mL). The combined organic layers are treated with TMT (3.2 g) and charcoal (6.4 g) at 70° C. for 3 h. The mixture is allowed to cool to 25° C. and filtered through celite. The filtrate is extracted twice with an aqueous K₂CO₃ solution (31.8 g of K₂CO₃ in 320 mL of water). The combined aqueous layers are used directly in the next step.
Step 2: To the above aqueous layer is added 2,4-dichlorophenethylamine (43.5 g). The resulting mixture is heated to 95° C. for 6 h. The mixture is cooled to 50° C. and n-BuOAc (150 mL) is added. The pH is adjusted to ˜4.94 with an aqueous solution of 4 M HCl (˜204 mL). The mixture is then cooled to 25° C. over 30 min and further cooled to 4° C. over 30 min. The mixture is filtered and the wet cake is washed with water (2×200 mL) and dried to afford 2-(3-{6-[2-(2,4-dichloro-phenyl)-ethylamino]-2-methoxy-pyrimidin-4-yl}-phenyl)-2-methyl-propionic acid (76.2 g, 97.5% purity by HPLC), which is further purified by recrystallization from DME/water to provide over 99% purity by HPLC (see FIG. 1).

### Example 2

### Dihydrogen Phosphate Salt of 2-(3-{6-[2-(2,4-dichlorophenyl)-ethylamino]-2-methoxypyrimidin-4-yl}-phenyl)-2-methylpropionic acid

A 1-L jacketed reactor is charged with 2-(3-{6-[2-(2,4-dichloro-phenyl)-ethylamino]-2-methoxy-pyrimidin-4-yl}-phenyl)-2-methylpropionic acid (50.0 g, 0.109 mol) and methanol (500 mL). The mixture is heated to 37.5±2° C. A 1:10 phosphoric acid:MeOH solution (110 mL) is then added to the mixture over 3 hrs. The mixture is then cooled to 10° C. and the temperature is maintained for 0.5 hrs. The solid is collected by filtration and the filter cake is washed with MeOH (120 mL) and dried to give 2-(3-{6-[2-(2,4-dichloro-phenyl)-ethylamino]-2-methoxy-pyrimidin-4-yl}-phenyl)-2-methylpropionic acid phosphoric acid salt (57.9 g, 95.6% yield). Gas Chromatography shows that the product contains 201 ppm residual methanol, well below the limit of ICH guideline of 3000 ppm.

The purity of the compound is analyzed by HPLC using the following conditions:

Instrument: Agilent 1100 series HPLC

### Column: Phenomenex Synergi 4 μHydro-RP, 150×4.6 mm

Conditions: Mobile phase: A: 0.1% TFA in acetonitrile; B: 0.1% TFA in water
Flow rate: 1.5 mL/min
Detector: 220 nm
Injection: 10 μL
Temperature: 25° C.
Run time: 18 min

### Gradient:

| Time (min) | Mobile Phase A | Mobile Phase B |
|---|---|---|
| 0 | 30 | 70 |
| 2.0 | 30 | 70 |
| 15.0 | 70 | 30 |
| 18.0 | 90 | 10 |

### Sample Preparation:

Dissolve testing sample in water/0.1% TFA in acetonitrile (20/80).

We claim:

1. A process for preparing 2-(3-{6-[2-(2,4-dichloro-phenyl)-ethylamino]-2-methoxy-pyrimidin-4-yl}-phenyl)-2-methyl-propionic acid of formula (A), 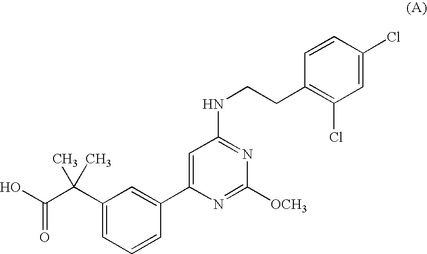
comprising:
(1) a coupling 4,6-dichloro-2-methoxy-pyrimidine with 3-(1-carboxy-1-methyl-ethyl)-phenyl boronic acid in the presence of a suitable palladium catalyst to provide 2-[3-(6-chloro-2-hydroxy-pyrimidin-4-yl)-phenyl]-2-methyl-propionic acid,
(2) coupling 2-[3-(6-chloro-2-methoxy-pyrimidin-4-yl)-phenyl]-2-methyl-propionic acid with 2,4-dichlorophenethylamine to provide 2-(3-{6-[2-(2,4-dichloro-phenyl)-ethylamino]-2-methoxy-pyrimidin-4-yl}-phenyl)-2-methyl-propionic acid.

2. The process according to claim 1, wherein the suitable palladium catalyst is Pd, PdCl₂, Pd(PPh₃)₄, pddf, or Pd(OAc)₂.

3. The process according to claim 1, wherein the suitable palladium catalyst is Pd(OAc)₂.

4. The process according to claim 3, wherein the coupling of 2-[3-(6-chloro-2-methoxy-pyrimidin-4-yl)-phenyl]-2-methyl-propionic acid with 2,4-dichlorophenethylamine is carried out in the presence of PPh₃.

5. The process according to claim 4, wherein the amount of Pd(OAc)₂ is about 0.01 mol % to about 0.5 mol %, and the amount of PPh₃ is about 0.02 mol % to about 1.0 mol %.

6. The process according to claim 4, wherein the amount of Pd(OAc)₂ is about 0.01 mol %, and the amount of PPh₃ is about 0.02 mol %.

7. The process according to claim 1, wherein the first step coupling reaction is carried out by adding 3-(1-carboxy-1-methyl-ethyl)-phenyl boronic acid to 4,6-dichloro-2-methoxypyrimidine over a period of time of about 2-6 hours.

8. The process according to claim 1, wherein the first step coupling reaction is carried out in the presence of a suitable base.

9. The process according to claim 8, wherein the suitable base is K₂CO₃.

10. The process according to claim 8, wherein the amount of the base is about 2 to 4 equivalents.

11. The process according to claim 8, wherein the amount of the base is about 3.0 equivalents.

12. The process according to claim 1, wherein the first step coupling reaction is carried out using about 1.0 to about 2.0 equivalents of 4,6-dichloro-2-methoxypyrimidine.

13. The process according to claim 1, wherein the first step coupling reaction is carried out using about 1.2 equivalents of 4,6-dichloro-2-methoxypyrimidine.

14. The process according to claim 1, wherein the first step coupling reaction is carried out in the presence of a suitable solvent system.

15. The process according to claim 14, wherein the suitable solvent system is a mixture of 1,2-dimethoxyethane and water.

16. The process according to claim 1, wherein before coupling 2-[3-(6-chloro-2-methoxy-pyrimidin-4-yl)-phenyl]-2-methyl-propionic acid with 2,4-dichlorophenethylamine, 2-[3-(6-chloro-2-hydroxy-pyrimidin-4-yl)-phenyl]-2-methylpropionic acid is purified by steps comprising:
removing excess 4,6-dichloro-2-methoxypyrimidne by a phase separation,
adjusting pH of the phase containing 2-[3-(6-chloro-2-methoxy-pyrimidin-4-yl)-phenyl]-2-methylpropionic acid to about 6.5 to about 7.5, and
extracting the phase containing 2-[3-(6-chloro-2-methoxy-pyrimidin-4-yl)-phenyl]-2-methylpropionic acid with a water-immiscible organic solvent.

17. The process according to claim 16 wherein the water-immiscible organic solvent is n-butyl acetate, ethyl acetate or toluene.

18. The process according to claim 1, wherein the palladium catalyst is removed from 2-[3-(6-chloro-2-methoxy-pyrimidin-4-yl)-phenyl]-2-methyl-propionic acid before coupling 2-[3-(6-chloro-2-methoxy-pyrimidin-4-yl)-phenyl]-2-methyl-propionic acid with 2,4-dichlorophenethylamine.

19. The process according to claim 18, wherein the palladium catalyst is removed by treating 2-[3-(6-chloro-2-methoxy-pyrimidin-4-yl)-phenyl]-2-methyl-propionic acid with TMT and charcoal.

20. The process according to claim 18, wherein the palladium catalyst is removed by treating 2-[3-(6-chloro-2-methoxy-pyrimidin-4-yl)-phenyl]-2-methyl-propionic acid with TMT and charcoal in an organic solvent at about 50-70° C.

21. The process according to claim 1, wherein 2-[3-(6-chloro-2-methoxy-pyrimidin-4-yl)-phenyl]-2-methyl-propionic acid is used directly in the second step as a solution.

22. The process according to claim 1, wherein the second step is carried out in water.

23. The process according to claim 1, further comprising purifying 2-(3-{6-[2-(2,4-dichlorophenyl)-ethylamino]-2-methoxypyrimidin-4-yl}-phenyl)-2-methylpropionic acid by recrystallization from an organic solvent and water.

24. The process according to claim 1, further comprising purifying 2-(3-{6-[2-(2,4-dichlorophenyl)-ethylamino]-2-methoxypyrimidin-4-yl}-phenyl)-2-methylpropionic acid by recrystallization from 1,2-dimethoxyethane and water.

25. A process for preparing dihydrogen phosphate salt of 2-(3-{6-[2-(2,4-dichlorophenyl)-ethylamino]-2-methoxypyrimidin-4-yl}-phenyl)-2-methylpropionic acid, comprising reacting 2-(3-{6-[2-(2,4-dichlorophenyl)-ethylamino]-2-methoxypyrimidin-4-yl}-phenyl)-2-methylpropionic acid with phosphoric acid in methanol.

26. A compound, which is 2-[3-(6-chloro-2-methoxypyrimidin-4-yl)-phenyl]-2-methylpropionic acid, or a salt thereof.

27. The compound according to claim 26, which is 2-[3-(6-chloro-2-methoxypyrimidin-4-yl)-phenyl]-2-methylpropionic acid.
